SEQUENCE LISTING (3 sequences)

SEQ ID NO: 1 - protein, 404 aa, Homo sapiens
MADKVLKEKRKLFIRSMGEGTINGLLDELLQTRVLNKEEMEKVKRENATVMDKTRALIDSVIPKGAQACQICITYICEEDSYLAGTLGLSADQTSGNYLNMQDSQGVLSSFPAPQAVQDNPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDSPGVVWFKDSVGVSGNLSLPTTEEFEDDAIKKAHIEKDFIAFCSSTPDNVSWRHPTMGSVFIGRLIEHMQEYACSCDVEEIFRKVRFSFEQPDGRAQMPTTERVTLTRCFYLFPGH

SEQ ID NO: 2 - protein, 88 aa, Homo sapiens
AIKKAHIEKDFIAFCSSTPDNVSWRHPTMGSVFIGRLIEHMQEYACSCDVEEIFRKVRFSFEQPDGRAQMPTTERVTLTRCFYLFPGH

SEQ ID NO: 3 - protein, 178 aa, Homo sapiens
NPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDSPGVVWFKD

(19) United States
(12) Patent Application Publication
(10) Pub. No.: US 2018/0259524 A1
(43) Pub. Date: Sep. 13, 2018

(54) METHODS FOR THE DETECTION AND MONITORING OF VASCULAR INFLAMMATION

(71) Applicant: UNIVERSITY OF CONNECTICUT, FARMINGTON, CT (US)

(72) Inventors: BRUCE LIANG, AVON, CT (US); LINDA SHAPIRO, MIDDLEBURY, CT (US); HECTOR LEONARDO AGUILA, WEST HARTFORD, CT (US); JU YONG LEE, AVON, CT (US); LIXIA YUE, UNIONVILLE, CT (US); MICHAEL AZRIN, SOUTHINGTON, CT (US)

(21) Appl. No.: 15/915,830

(22) Filed: Mar. 8, 2018

Related U.S. Application Data
(60) Provisional application No. 62/469,105, filed on Mar. 9, 2017.

(51) Int. Cl. G01N 33/573

(52) U.S. Cl. CPC G01N 33/573; G01N 2800/32; G01N 2800/52; G01N 2800/324; G01N 2333/96466

(57) ABSTRACT

Disclosed herein are methods of detecting vascular inflammation associated with acute myocardial infarction and/or prognosticating acute myocardial infarction by detecting a proteolytic fragment of caspase-1 such as the p20 fragment.

### CROSS-REFERENCE TO RELATED APPLICATIONS

This application claims priority to U.S. Provisional Application 62/469,105 filed on Mar. 9, 2017, which is incorporated herein by reference in its entirety.

### BACKGROUND

Vascular inflammation plays an important role in the pathophysiology of acute myocardial infarction (AMI), contributing to progression of atherosclerosis and the processes leading to AMI. Vascular inflammation is also critical in infarct healing where phagocytosis of damaged tissues and its resolution is needed for subsequent collagen formation and angiogenesis. Pro-inflammatory monocytes and macrophages have been implicated as significant contributors to accelerated atherosclerotic plaque formation and its instability. Similarly, the inflammatory monocytes and macrophages are thought to play a detrimental role in wound healing and remodeling of the post-infarction heart. Finally, persistent vascular inflammation may play a role in recurrent MI after the initial AMI.

There remains a need for new biomarkers suitable for detecting and/or diagnosing vascular inflammation.

### SUMMARY

In one embodiment, a method for detecting vascular inflammation in an individual comprises providing a serum or plasma sample from the individual; directly or indirectly measuring a level of a caspase-1 proteolytic fragment in the plasma or serum sample from the individual; comparing the level of the caspase-1 proteolytic fragment in the plasma or serum sample from the individual to a control level in a control sample; and detecting vascular inflammation when the level of the caspase-1 proteolytic fragment in the plasma or serum sample is elevated by 20% or more compared to the control level in the control sample, wherein the individual is in need of detection of vascular inflammation.

In another embodiment, a prognostic method for increased mortality and/or morbidity resulting from acute myocardial infarction in an individual, and/or predicting the recurrence of myocardial infarction in the individual comprises providing a serum or plasma sample from the individual; directly or indirectly measuring a level of a caspase-1 proteolytic fragment in the plasma or serum sample from the individual; comparing the level of the caspase-1 proteolytic fragment in the plasma or serum sample from the individual to a reference population or a control level in a control sample from healthy subjects, and detecting an increased risk of mortality and/or morbidity resulting from acute myocardial infarction wherein, for example, the level of the caspase-1 proteolytic fragment in the serum or plasma sample is in the top tertile or quartile of a patient population or wherein the level of the caspase-1 proteolytic fragment in the serum or plasma sample is increased by 20% or more compared to the control level in healthy subjects, wherein the individual has had an acute myocardial infarction.

In yet another embodiment, a method for determining the success of a therapy for acute myocardial infarction in an individual comprises providing a serum or plasma sample from the individual; directly or indirectly measuring a level of a caspase-1 proteolytic fragment in the plasma or serum sample, directly or indirectly measuring a level of a caspase-1 proteolytic fragment in the plasma or serum sample from the individual; and detecting an increased risk of mortality and/or morbidity resulting from acute myocardial infarction in the individual wherein the level of the caspase-1 proteolytic fragment in the serum or plasma sample is in the top tertile or quartile of a reference patient population or wherein the level of the caspase-1 proteolytic fragment in the serum or plasma sample is increased by 20% or more compared to the control level from healthy subjects, wherein the individual has been treated for an acute myocardial infarction.

### BRIEF DESCRIPTION OF THE FIGURES

FIG. 1 shows Caspase-1 p20 peptide levels during and after acute STEMI. Serum samples were obtained from patients with type 1 acute myocardial infarction including both STEMI and NSTEMI subjects as well as from healthy subjects. The serum p20 peptide levels were measured as described in Methods. The non-peak and peak levels during the acute event, the post-MI level, and the level from healthy subjects were presented as median and interquartile range. *P<0.05 healthy level is lower than non-peak, peak, and post-MI levels; **P<0.05 peak level is higher than that of any other group.

FIG. 2 shows a schematic of a proposed pathway of activation for Caspase-1 by NLRP3. NLRP3—Nod-like receptor protein 3 or nucleotide-binding domain, leucine-rich-containing family, pyrin domain-containing 3, NBD—Nucleotide-binding domain, LRR—Leucine-rich repeat, PYD—pyrin domain, CARD—Caspase recruitment domain, ASC—Apoptosis-associated Speck-like protein containing a CARD.

FIG. 3 shows the correlation between average peak Caspase p20 level by tertile and age. T1: N=63, Mean age=62. T2: N=56, Mean age=64.3. T3: N=51, Mean age=66.9. P=0.01, Linear regression. Error bars are SEM.

FIG. 4 shows the difference in peak caspase-1 p20 concentrations in STEMI subjects between patients with and without hypertension. P=0.0056, Wilcoxon, N=31 per group. Error bars are SEM.

FIG. 5 shows the trend in post-MI left ventricular fractional shortening with peak Caspase-1 p20 concentration during AMI. r=−0.35, P=0.05, N=31.

### DETAILED DESCRIPTION

Increased vascular inflammation is associated with vascular disease and contributes to the pathogenesis of acute myocardial infarction (AMI). Ongoing inflammation likely also plays a role in the recurrent MI after the initial AMI. The inflammasome is a protein complex in the cytoplasm of monocytes and macrophages that mediates the inflammatory response to pathogen-associated molecular patterns (PAMPs) or danger-associated molecular patterns (DAMPs). Cells dying from ischemic injury release intracellular DAMPs to initiate the innate inflammatory response. These molecules bind to the immune Pattern Recognition Receptors (PPRs) on the cell surface to activate the inflammasome in the cytoplasm of cells such as monocytes, macrophages and fibroblasts. Similarly, in vascular disease, inflammasome activation by DAMPs results in a more severe ischemia/reperfusion injury as well as adversely impacting tissue repair. The NLRP3 inflammasome is a cytoplasmic protein complex containing pro-caspase-1 and the ASC protease (apoptosis speck-like protein caspase-recruitment domain), which cleaves pro-caspase-1 into its active p20 and p10 dimers. Activated caspase-1 cleaves pro-IL-1β and pro-IL18 to active IL-1 β and IL-18 respectively. IL-1 β is secreted from monocytes, macrophages, and cardiac fibroblasts and functions to recruit and activate inflammatory leukocytes and delay transdifferentiation of fibroblasts to myofibroblasts.

While there has been growing pre-clinical evidence for an important role of the NLRP3 inflammasome in atherosclerosis, ischemia/reperfusion injuries, and post-infarction remodeling in animals, very little is known regarding this inflammasome in patients with vascular disease. The inventors herein have unexpectedly discovered that cleaved caspase-1 products can be quantified in human serum and plasma and that it is a marker for vascular inflammation in patients with vascular disease. Cleaved caspase-1 fragments can be used to determine the extent of vascular inflammation before and after AMI and can be used to predict the post-AMI prognosis as well as to determine the success of therapy for acute myocardial infarction.

Caspases are a family of proteases involved in the regulation of controlled cell death and inflammation. Caspases are most recognized for the induction of apoptosis, a non-inflammatory form of cell death; however, caspases are also involved in several types of cell death, some of which can be inflammatory. Caspases can be found in pro-inflammatory monocytes and macrophages and play a role in the remodeling of the heart after an infarction.

Caspase-1 is known as a pro-inflammatory caspase and has been shown to be responsible for the activation of inflammatory cytokines and the initiation of pyroptosis, an inflammatory type of cell death induced by intracellular pathogen markers. Caspase-1 specifically converts the inactive forms of the cytokines IL-1β and IL-18 to the active forms. Active IL-1β acts as a chemokine to recruit inflammatory cells to the site of release. Active IL-18 plays an important role in the production of IFN-γ and enhances the cytolytic activity of natural killer cells.

Caspase-1 (Uniprot Accession No. P29466, SEQ ID NO:1) is synthesized as an inactive proenzyme procaspase-1 that is cleaved into the active caspase-1 fragments p10 (nucleotides 317-404, SEQ ID NO:2) and p20 (nucleotides 120-297, SEQ ID NO:3). Disclosed herein are methods of detecting and/or prognosticating vascular inflammation by detecting a proteolytic fragment of caspase-1 such as the p20 peptide or the p10 fragment.

In one embodiment, a method for detecting vascular inflammation in an individual comprises providing a serum or plasma sample from the individual, and directly or indirectly measuring a level of a caspase-1 proteolytic fragment in the plasma or serum sample from the individual. In one embodiment, the individual is experiencing or has previously experienced an acute myocardial infarction. When the individual has previously experienced a myocardial infarction, the method can comprise predicting the recurrence of myocardial infarction in the individual. The acute myocardial infarction can be an ST segment elevation myocardial infarction (STEMI) or a non-ST segment elevation myocardial infarction (NSTEMI).

As used herein, a plasma sample is a blood sample in which blood cells have been removed from the sample. A serum sample is similar to a plasma sample except the clotting factors have also been removed.

In an embodiment, the individual is experiencing inflammation in an organ that can be damaged via systemic vascular inflammation, wherein the organ is not the heart. For example, the brain or the endothelium or kidney may be damaged by systemic inflammation.

The method can further include comparing the level of the caspase-1 proteolytic fragment in the sample to a control level in a control sample. Exemplary control samples include samples from a normal population, samples from control individual when the control individual is not experiencing vascular inflammation, or from an individual experiencing an acute myocardial infarction.

In another embodiment, the method further comprises detecting vascular inflammation in the individual when the level of the caspase-1 proteolytic fragment in the plasma or serum sample from the individual is substantially elevated compared to the control level, wherein the control level is measured from a serum sample when the individual is not experiencing vascular inflammation. Substantially elevated includes being elevated by 20% or more compared to the control level in the control sample.

In a specific aspect, a method for detecting vascular inflammation in an individual comprises

providing a serum or plasma sample from the individual,

directly or indirectly measuring a level of a caspase-1 proteolytic fragment in the plasma or serum sample from the individual,

comparing the level of the caspase-1 proteolytic fragment in the plasma or serum sample from the individual to a control level in a control sample, and

detecting vascular inflammation in the individual, when the level of the caspase-1 proteolytic fragment in the plasma or serum sample from the individual is elevated by 20% or more compared to the control level in the control sample,

wherein the individual is in need of detection of vascular inflammation.

In another aspect, a prognostic method for increased mortality and/or morbidity resulting from acute myocardial infarction in an individual, and/or predicting the recurrence of myocardial infarction in the individual comprises

providing a serum or plasma sample from the individual,

directly or indirectly measuring a level of a caspase-1 proteolytic fragment in the plasma or serum sample from the individual,

comparing the level of the caspase-1 proteolytic fragment in the plasma or serum sample from the individual to a reference population or a control level in a control sample from healthy subjects, and

detecting an increased risk of mortality and/or morbidity resulting from acute myocardial infarction in the individual wherein the level of the caspase-1 proteolytic fragment in the serum or plasma sample is in the top tertile or quartile of the reference patient population, or wherein the level of the caspase-1 proteolytic fragment in the serum or plasma sample is increased by 20% or more compared to the control level from healthy subjects,

wherein the individual has had an acute myocardial infarction.

In yet another aspect, a method for determining the success of a therapy for acute myocardial infarction in an individual, comprises

providing a serum or plasma sample from the individual,

directly or indirectly measuring a level of a caspase-1 proteolytic fragment in the plasma or serum sample from the individual,

comparing the level of the caspase-1 proteolytic fragment in the plasma or serum sample from the individual to a reference population or a control level in a control sample from healthy subjects, and

detecting an increased risk of mortality and/or morbidity resulting from acute myocardial infarction in the individual wherein the level of the caspase-1 proteolytic fragment in the serum or plasma sample is in the top tertile or quartile of a reference patient population or wherein the level of the caspase-1 proteolytic fragment in the serum or plasma sample is increased by 20% or more compared to the control level from healthy subjects,

wherein the individual has been treated with therapy for an acute myocardial infarction.

As used herein, a reference patient population is generally a healthy population. Is this correct?

Caspase-1 (SEQ ID NO:1) has the sequence:

MADKVLKEKR KLFIRSMGEG TINGLLDELL QTRVLNKEEM
EKVKRENATV MDKTRALIDS VIPKGAQACQ ICITYICEED
SYLAGTLGLS ADQTSGNYLN MQDSQGVLSS FPAPQAVQDN
PAMPTSSGSE GNVKLCSLEE AQRIWKQKSA EIYPIMDKSS
RTRLALIICN EEFDSIPRRT GAEVDITGMT MLLQNLGYSV
DVKKNLTASD MTTELEAFAH RPEHKTSDST FLVFMSHGIR
EGICGKKHSE QVPDILQLNA IFNMLNTKNC PSLKDKPKVI
IIQACRGDSP GVVWFKDSVG VSGNLSLPTT EEFEDDAIKK
AHIEKDFIAF CSSTPDNVSW RHPTMGSVFI GRLIEHMQEY
ACSCDVEEIF RKVRFSFEQP DGRAQMPTTE RVTLTRCFYL FPGH

The p10 fragment is amino acids 317-404 (SEQ ID NO:2), and has the sequence:

AIKK AHIEKDFIAF CSSTPDNVSW RHPTMGSVFI GRLIEHMQEY
ACSCDVEEIF RKVRFSFEQP DGRAQMPTTE RVTLTRCFYL FPGH

The p20 fragment is amino acids 120-297 (SEQ ID NO: 3) and has the sequence:

N PAMPTSSGSE GNVKLCSLEE AQRIWKQKSA EIYPIMDKSS
RTRLALIICN EEFDSIPRRT GAEVDITGMT MLLQNLGYSV
DVKKNLTASD MTTELEAFAH RPEHKTSDST FLVFMSHGIR
EGICGKKHSE QVPDILQLNA IFNMLNTKNC PSLKDKPKVI
IIQACRGDSP GVVWFKD

The term “level” relates to amount or concentration of a peptide or polypeptide in a patient or a sample taken from a patient. The term “measuring” accordingly relates to determining the amount or concentration, preferably semi-quantitatively or quantitatively, of the nucleic acid, peptide, polypeptide, or other substance of interest. Measuring can be done directly or indirectly. Indirect measuring includes measuring of cellular responses, bound ligands, labels, or enzymatic reaction products. In one embodiment, indirect measuring includes the use of an antibody.

The amount, level or presence of a caspase-1 proteolytic fragment is determined using any of a variety of techniques known to the skilled artisan such as, for example, a technique selected from the group consisting of, immunohistochemistry, immunofluorescence, an immunoblot, a Western blot, a dot blot, an enzyme linked immunosorbent assay (ELISA), radioimmunoassay (MA), enzyme immunoassay, fluorescence resonance energy transfer (FRET), matrix-assisted laser desorption/ionization time of flight (MALDI-TOF), electrospray ionization (ESI), mass spectrometry (including tandem mass spectrometry, e.g. LC MS/MS), surface-enhanced laser desorption ionization mass spectrometry (SELDI-MS), biosensor technology, evanescent fiber-optics technology or protein chip technology.

In one embodiment the assay used to determine the amount or level of caspase-1 or a fragment thereof is a semi-quantitative assay. In another embodiment the assay used to determine the amount or level of caspase-1 or a fragment thereof is a quantitative assay. As will be apparent from the preceding description, such an assay may require the use of a suitable control, e.g., from a normal individual or matched normal control.

Standard solid-phase ELISA or FLISA formats are particularly useful in determining the concentration of a protein from a variety of samples.

Caspase-1 or a fragment thereof can be measured in the body fluid sample, e.g., serum or plasma, using assays that detect caspase-1 or a fragment thereof, for example, radioisotopic immunoassays or non-isotopic immunoassays, e.g., fluorescent immunoassays, enzymatic immunoassays, such as an enzyme linked immunoassay (ELISA).

By way of example, other means for determining and measuring caspase-1 or a fragment thereof in a sample include affinity chromatography, ligand binding assays and lectin binding assays. Immunoassays, especially non-radioisotopic immunoassays, are particularly suitable. Normal range and normal mean values can be determined for the assay being carried out based on normal (healthy) population samples, as is known and practiced in the art.

As used herein, the term antibody includes polyclonal and monoclonal antibodies of any isotype (IgA, IgG, IgE, IgD, IgM), or an antigen-binding portion thereof, including but not limited to F(ab) and Fv fragments, single chain antibodies, chimeric antibodies, humanized antibodies, and a Fab expression library. Antibodies useful as detector and capture antibodies may be prepared by standard techniques well known in the art.

Antibodies generated against caspase-1 or a fragment thereof, can be obtained by direct injection of an immunogenic caspase-1 preparation into an animal, or by administering all, or the caspase-1 p-20 polypeptides to an animal, preferably a nonhuman animal. For the preparation of monoclonal antibodies, a suitable technique which provides antibodies produced by continuous cell line cultures can be used. Examples include the hybridoma technique, the trioma technique, the human B-cell hybridoma technique, and the EBV-hybridoma technique to produce human monoclonal antibodies. Techniques described for the production of single chain antibodies can be adapted to produce single chain antibodies to caspase-1. Also, transgenic mice may be used to express humanized antibodies to immunogenic caspase-1 or a fragment thereof.

Methods for producing and screening for antibodies to caspase-1 or a fragment thereof using hybridoma technology are routine and well known in the art. In a nonlimiting example, mice can be immunized with an immunogen, caspase-1 or a fragment thereof, or with a cell expressing these polypeptides or peptides. Once an immune response is detected, e.g., antibodies specific for the antigen are detected in the sera of immunized mice, the spleen is harvested and splenocytes are isolated. The splenocytes are then fused by well-known techniques to any suitable myeloma cells available from the ATCC. Hybridomas are selected and cloned by limiting dilution techniques. The hybridoma clones are then assayed by methods known in the art to determine and select those cells that secrete antibodies capable of binding to caspase-1 or a fragment thereof. Ascites fluid, which generally contains high levels of antibodies, can be generated by injecting mice with positive hybridoma clones.

Caspase-1 or a fragment thereof comprising one or more immunogenic caspase-1 epitopes which elicit an antibody response can be introduced together with a carrier protein, such as an albumin, to a host animal (such as rabbit, mouse, rat, sheep, or goat). Alternatively, if the polypeptide is of sufficient length (e.g., at least about 25 amino acids), the polypeptide can be presented without a carrier. However, immunogenic epitopes comprising as few as 5 to 10 amino acids have been shown to be sufficient to raise antibodies capable of binding to, at the very least, linear epitopes in a denatured polypeptide (e.g., in Western blotting).

Caspase-1 or a fragment thereof can be used to induce antibodies according to methods well known in the art including, but not limited to, in vivo immunization, in vitro immunization, and phage display methods. If in vivo immunization is used, animals can be immunized with free peptide; however, the anti-peptide antibody titer may be boosted by coupling the peptide to a macromolecular carrier, such as keyhole limpet hemacyanin (KLH), or tetanus toxoid (TT). For instance, peptides containing cysteine residues can be coupled to a carrier using a linker such as maleimidobenzoyl-N-hydroxysuccinimide ester (MBS), while other peptides may be coupled to carriers using a more general linking agent, such as glutaraldehyde.

Antibodies specific for caspase-1 or a fragment thereof are produced by methods known in the art for the synthesis of antibodies, in particular, by chemical synthesis, by intracellular immunization (i.e., intrabody technology), or by recombinant expression techniques. Methods of producing antibodies include, but are not limited to, hybridoma technology, EBV transformation, as well as through the use recombinant DNA technology. Recombinant expression of an antibody, or a fragment, derivative, variant or analog thereof, (e.g., a heavy or light chain of an anti-caspase-1 antibody), requires construction of an expression vector containing a polynucleotide that encodes the antibody. Once a polynucleotide encoding an antibody molecule or a heavy or light chain of an antibody, or portion thereof (preferably containing the heavy or light chain variable domain) has been obtained, the vector for the production of the antibody molecule can be produced by recombinant DNA technology using techniques well known in the art. In vitro recombinant DNA techniques, synthetic techniques, and in vivo genetic recombination methods, which are well known to those skilled in the art, can be used to construct expression vectors containing antibody coding sequences and appropriate transcriptional and translational control signals. Such vectors can include the nucleotide sequence encoding the constant region of the antibody molecule and the variable region of the antibody cloned into such a vector for expression of the entire heavy or light chain.

The expression vector is then introduced into a host cell by conventional techniques and the transfected cells are cultured by conventional techniques to produce an anti-capase-1 antibody. A variety of host expression vector systems can be utilized to express the antibody molecules. Such expression systems represent vehicles by which the coding sequences of interest can be expressed, their encoded products produced and subsequently purified. These systems also represent cells which can, when transformed or transfected with the appropriate nucleotide coding sequences, express an antibody molecule of the invention in situ. Cell expression systems include, but are not limited, to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces or Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus (CaMV) or tobacco mosaic virus (TMV)), transformed with recombinant plasmid expression vectors (e.g., Ti plasmid) containing antibody coding sequences; or mammalian cell systems (e.g., COS, CHO, BHK, 293, 3T3, NSO cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Specifically, bacterial cells such as E. coli, and more specifically, eukaryotic cells, especially for the expression of whole recombinant antibody molecules, are used for the expression of a recombinant antibody molecule. For example, mammalian cells such as Chinese hamster ovary (CHO) cells, in conjunction with a vector such as the major intermediate early gene promoter element from human cytomegalovirus, is an effective expression system for antibody production.

Once an anti-caspase-1 antibody has been produced by an animal, chemically synthesized, or recombinantly expressed, it can be purified by methods known in the art for the purification of an immunoglobulin or polypeptide molecule, for example, by chromatography (e.g., ion exchange, affinity, particularly by affinity for the specific antigen, Protein A, and sizing column chromatography), centrifugation, differential solubility, or by any other standard technique for the purification of proteins.

In one embodiment, an ELISA assay initially involves preparing an antibody specific to caspase-1 or a fragment thereof, preferably a monoclonal antibody. In addition, a reporter antibody is used. In some ELISA protocols, the reporter antibody recognizes and binds to the caspase-1 specific monoclonal antibody. To the reporter antibody is attached a detectable reagent such as a radioactive isotope, a fluorescent moiety, a chemiluminescent moiety, or, in an ELISA, an enzyme, such as horseradish peroxidase or alkaline phosphatase.

As is appreciated by those skilled in the art, ELISAs can be performed in a number of assay formats. In one ELISA format, a host sample, e.g., a patient body fluid sample, is incubated on a solid support, e.g., the wells of a microtiter plate, or a polystyrene dish, to which the proteins in the sample can bind. Any free protein binding sites on the dish are then blocked by incubating with a non-specific protein such as bovine serum albumin. The monoclonal antibody is then added to the solid support, e.g., the wells or the dish, and allowed to incubate. During the incubation time, the monoclonal antibodies attach to any caspase-1 polypeptides that have attached to the polystyrene dish.

Unbound monoclonal antibody is washed away using an appropriate buffer solution. The reporter antibody, e.g., linked to horseradish peroxidase, is added to the support, thereby resulting in the binding of the reporter antibody to any monoclonal antibody which has bound to caspase-1 or a fragment thereof present in the sample. Unattached reporter antibody is then washed away. Peroxidase substrate is added to the support and the amount of color developed in a given time period provides a measurement of the amount of caspase-1 or a fragment thereof that is present in a given volume of patient sample when compared to a standard curve.

In another ELISA format, antibody specific for a particular analyte is attached to the solid support, i.e., the wells of a microtiter plate or a polystyrene dish, and a sample containing analyte is added to the substrate. Detectable reporter antibodies, which bind to the analyte that has bound to the capture antibodies on the support, are then added, after the appropriate incubations and washings, and analyte-antibody complexes are detected and quantified.

In one embodiment, the ELISA assay is a sandwich type ELISA immunoassay typically performed using microtiter plates. A capture antibody, that can be polyclonal or monoclonal, preferably a monoclonal antibody, that specifically recognizes an epitope in the extracellular portion caspase-1 or a fragment thereof is used, along with a labeled detector antibody, e.g., an alkaline phosphatase-labeled antibody, or a horse radish peroxidase-labeled antibody, preferably a monoclonal antibody. The detector antibody also specifically recognizes an epitope on the extracellular protein domain of caspase-1 or a fragment thereof. Preferably, the capture antibody does not inhibit binding to caspase-1 or a fragment thereof. The production of both polyclonal and monoclonal antibodies, particularly monoclonal antibodies that are specific for caspase-1 or a fragment thereof, is performed using techniques known in the art.

In a particular embodiment, a capture anti-caspase-1 antibody of the assay method, is immobilized on the interior surface of the wells of the microtiter plate. To perform the assay, an appropriate volume of sample is incubated in the wells to allow binding of the antigen by the capture antibody. The immobilized antigen is then exposed to the labeled detector antibody. Addition of substrate to the wells, if the detectable label is alkaline phosphatase, for example, allows the catalysis of a chromogen, i.e., para-nitrophenylphosphate (pNPP), if the label is alkaline phosphatase, into a colored product. The intensity of the colored product is proportional to the amount of caspase-1 that is bound to the microtiter plate.

Standards are used to allow accurate quantitative determinations of caspase-1 or a fragment thereof in the samples undergoing analysis. A microtiter plate reader simultaneously measures the absorbance of the colored product in the standard and the sample wells. Correlating the absorbance values of samples with the standards run in parallel in the assay allows the determination of the levels of caspase-1 or a fragment thereof in the sample. Samples are assigned a quantitative value of caspase-1 or a fragment thereof in nanograms per milliliter (ng/ml) of serum, plasma, other body fluid, or cell culture fluid.

For ease and simplicity of detection, and its quantitative nature, an exemplary assay is the sandwich or double antibody assay of which a number of variations exist. In one embodiment of a sandwich assay, unlabeled antibody is immobilized on a solid phase, e.g., a microtiter plate, and the sample to be tested is added. After a certain period of incubation to allow formation of an antibody-antigen complex, a second antibody, labeled with a reporter molecule capable of inducing a detectable signal, is added and incubation is continued to allow sufficient time for binding with the antigen at a different site, resulting with a formation of a complex of antibody-antigen-labeled antibody. The presence of the antigen is determined by observation of a signal which may be quantitated by comparison with control samples containing known amounts of antigen.

Alternatively, the amount of caspase-1 or a fragment thereof is detected using a radioimmunoassay (MA). The basic principle of the assay is the use of a radiolabeled antibody or antigen to detect antibody-antigen interactions. An antibody or ligand that specifically binds to a protein is bound to a solid support and a sample brought into direct contact with said antibody. To detect the level of bound antigen, an isolated and/or recombinant form of the antigen is radiolabeled and brought into contact with the same antibody. Following washing, the level of bound radioactivity is detected. As any antigen in the biological sample inhibits binding of the radiolabeled antigen the level of radioactivity detected is inversely proportional to the level of antigen in the sample. Such an assay may be quantitated by using a standard curve using increasing known concentrations of the isolated antigen.

In another embodiment, Western blotting is used to determine the level of caspase-1 or a fragment thereof in a sample. In such an assay protein from a sample is separated using sodium doedecyl sulphate polyacrylamide gel electrophoresis (SDS-PAGE) using techniques known in the art. Separated proteins are then transferred to a solid support, such as, for example, a membrane (e.g., a PVDF membrane), using, for example, electrotransfer. This membrane is then blocked and probed with a labeled antibody or ligand that specifically binds to caspase-1 or a fragment thereof. Alternatively, a labeled secondary, or even tertiary, antibody or ligand is used to detect the binding of a specific primary antibody. The level of label is then determined using an assay appropriate for the label used.

In other embodiments, the detection of the level of caspase-1 or a fragment thereof is done by a method such as, for example, mass spectrometry, matrix-assisted laser desorption/ionization time of flight (MALDI-TOF), electrospray ionisation (ESI), protein chip, biosensor technology, or fluorescence resonance energy transfer.

Biosensor devices generally employ an electrode surface in combination with current or impedance measuring elements to be integrated into a device in combination with the assay substrate (such as that described in U.S. Pat. No. 5,567,301). An antibody/ligand that specifically binds to a protein of interest is preferably incorporated onto the surface of a biosensor device and a biological sample contacted to said device. A change in the detected current or impedance by the biosensor device indicates protein binding to said antibody. Some forms of biosensors known in the art also rely on surface plasmon resonance to detect protein interactions, whereby a change in the surface plasmon resonance surface of reflection is indicative of a protein binding to a ligand or antibody (U.S. Pat. Nos. 5,485,277 and 5,492,840).

Biosensors are of particular use in high throughput analysis due to the ease of adapting such systems to micro- or nano-scales. Furthermore, such systems are conveniently adapted to incorporate several detection reagents, allowing for multiplexing of diagnostic reagents in a single biosensor unit. This permits the simultaneous detection of several proteins or peptides in a small amount of body fluid.

The data presented herein clearly show that a cleaved caspase-1 product, the caspase-1 p20 fragment, is detectable in human circulation. The caspase-1 p20 level is increased in patients with vascular inflammation, including both STEMI and NSTEMI subjects. Thus, the level of caspase-1 proteolytic fragment can be used as a novel marker for vascular inflammation.

Elevated levels of caspase-1 proteolytic fragments in AMI patients indicate vascular inflammation. The caspase-1 proteolytic fragment levels serve as a prognostic indicator for increased mortality and/or morbidity and may also predict recurrence of another MI subsequently. By substantially elevated, it is meant that the level is increased by 20% or more, specifically 50% or more, and more specifically 100% or more.

The disclosure is further illustrated by the following non-limiting examples.

### Examples

### Example 1: Serum Caspase-1 p20 Levels During and after Acute MI

### Methods

The study population consisted of 198 consecutive enrolled type I MI patients, including both STEMI and NSTEMI subjects, for whom complete data for caspase-1 were available during the acute event and the post-MI period between Dec. 10, 2007 and Dec. 20, 2017. Acute MI indicated both STEMI and NSTEMI. All STEMI patients underwent primary percutaneous coronary intervention upon admission and received subsequent clinical care in a standard manner. STEMI was defined as characteristic symptoms of myocardial ischemia in association with persistent electrocardiographic (ECG) ST segment elevation ≥0.1 mV in unipolar leads or ≥0.2 mV in the anterior leads and subsequent release of biomarkers of myocardial necrosis with a cTnI above 0.05 μg/l associated with a rise or fall during subsequent sampling. cTnI, creatine kinase (CK) and CK-MB were measured at admission and then every 8-12 hours for 24 hours. Peak levels were ascertained from the serial samples and were determined as the highest level if there was at least one lower value before and after the peak value. Thirty eight of the 78 subjects had a nonpeak level drawn before the peak level. Informed consent was obtained from all subjects with a protocol that was approved by Institutional Review Board at the University of Connecticut Health Center.

Caspase-1 20 kDa Peptide and MI Biomarker Measurements:

Upon activation, the inactive procaspase-1 is cleaved into the active caspase-1 consisting of p10 and p20 subunits. The cleaved p20 fragment in serum was detected using a commercial sandwich ELISA assay (Human Caspase-1/ICE Quantikine© ELISA kit from R&D Systems). Serum samples were stored at −80° C. after their fresh isolation. Serum was diluted 1:1 in sample diluent buffer, incubated for 1.5 hours at room temperature, then measured on a 96-well microplate coated with caspase-1 antibody. The amount of p20 was determined by spectrophotometric analysis at 450 and 540 nm (OD₄₅₀ and OD₅₄₀ respectively) using a Multi-Detection Microplate Reader (Biotek, Model Synergy 2, Winooski, Vt.) according to the protocol provided with the assay. Optical density (OD) measures the transmitted radiant power of sample obtained from each plate in comparison to a standard curve consisting of known caspase-1 p20 levels. This ratio was then used to derive pg/mL units, which was done to correct for any inconsistencies in the timing of reagents that may have occurred in each assay. The p20 levels were measured at least twice 8 hours apart during the initial 24 hours after admission and were considered to be acute MI values. Peak p20 levels were ascertained retrospectively from the serial samples and were determined as the highest level of the two initial blood draws upon admission to the hospital. A subsequent measurement was made up to 90 days after the acute MI and is considered the post-MI value.

CTnI was determined by the Access AccuTnI assay as previously described (15). CK-MB was determined by the Access CK-MB assay and both cTnI and CK-MB (μg/L) levels were measured using Beckman DXI 800. The 99^{th} percentile value was used as the upper reference limit.

Cardiac Catheterization and Echocardiography:

LV ejection fraction (LVEF) was obtained by visual inspection from echocardiography within 24 hours of admission to the hospital. Percutaneous coronary intervention was performed with a door-to-balloon time of less than 90 minutes in STEMI patients and within the indexed hospital stay in Non-STEMI patients.

Statistical Analyses:

Medians with interquartile ranges for non-normally distributed variables were used to describe continuous variables. Given that cTnI, CK, CK-MB and p20 values are not normally distributed, correlations were calculated using Spearman rank correlation. The non-parametric Wilcoxon Signed Rank test was used for paired comparisons between peak acute MI p20 and post-MI values. For analysis of difference between peak acute MI p20 value or post-MI p20 value and p20 value of healthy control subjects, Mann-Whitney test was used. For all analyses, a P value of less than 0.05 was considered significant. All P values reported were two-sided. Statistical analyses were performed with GraphPad Prism version 6.00 for Windows, GraphPad Software, La Jolla Calif. USA, www.graphpad.com.

### Results:

Patients. In a series of study from Dec. 10, 2007 to Oct. 23, 2017, a total of 198 patients were enrolled. Not all subjects had the non-peak, peak or post-MI samples that were non-hemolyzed. Eighty subjects had complete set of non-peak, peak and post-MI caspase-1 assayed including 35 STEMI and 45 NSTEMI patietns.STEMI patients had occluded infarct-related artery with TIMI 0 or 1 grade flow. Primary percutaneous coronary intervention (PCI) was performed with successful reperfusion in all STEMI subjects. Post-PCI Thrombolysis In Myocardial Infarction (TIMI) flow was grade 3 in all STEMI patients. Nearly all NSTEMI patients also received successful PCI. There were no complications such as stroke, renal failure, cardiogenic shock or need for coronary artery bypass surgery. Baseline clinical characteristics of the study population are shown in Table 1.

Serum Caspase-1 p20 Levels During and after Acute MI

There was a rise and fall of p20 during and post-MI (FIG. 1). For the entire MI group that includes both STEMI and NSTEMI patients, the peak acute MI p20 levels (171.7 pg/ml±11.06 pg/ml, n=80) were significantly higher than post-MI levels (116.4 pg/ml±8.2 pg/ml, n=80, p=<0.0001). Both peak acute and post-MI levels were higher than healthy control patients (79.19 pg/ml±5.3 pg/ml, n=91, healthy). The non-peak (100.7 pg/ml±6.6 pg/ml) level obtained during the acute MI was also higher than that of the healthy controls. Consistent with a rise of the caspase-1 level during the acute MI phase, the peak acute level was higher than the non-peak acute level.

In analyzing those with NSTEMI, the peak acute MI level (180.9 pg/ml±15.82 pg/ml) was higher than post-MI level which was 114.3 pg/ml±12.36 pg/ml (n=45, P<0.01). Both peak acute and post-MI levels were also higher than healthy control patients (peak acute MI vs. healthy p=<0.001, post-MI vs. healthy p<0.01). The peak acute level was higher than the non-peak acute level. The non-peak level was similar to that of post-MI level, consistent with a fall of the caspase-1 p20 level even during the acute phase of NSTEMI.

For the STEMI group, peak acute level (159.9 pg/ml±15.01 pg/ml) was higher than the non-peak acute level (103.4 pg/ml±8.43 pg/ml). The acute peak and non-peak levels as well as that of post-MI (119.1 pg/ml±10.1 pg/ml) were all higher than that of the healthy control level (Nonpeak vs. Healthy p<0.01, Peak vs Healthy p=<0.001, Post vs. Healthy p=<0.001). The acute peak level was higher than the post-MI levels (P<0.05; Friedman test and post-test comparison). The non-peak level was similar to that of post-MI level, consistent with a rise and a fall of the caspase-1 p20 level during the acute phase of STEMI. The peak p20 level during the acute MI was similar in NSTEMI was similar to that in STEMI patients.

Correlations with biomarkers for acute MI. In all subjects, the peak cTnI, CK, and CK-MB values were obtained within 24 hours after reperfusion. There was no correlation of peak acute MI p20 with peak CK, CK-MB or troponin by Spearman testing.

### DISCUSSION

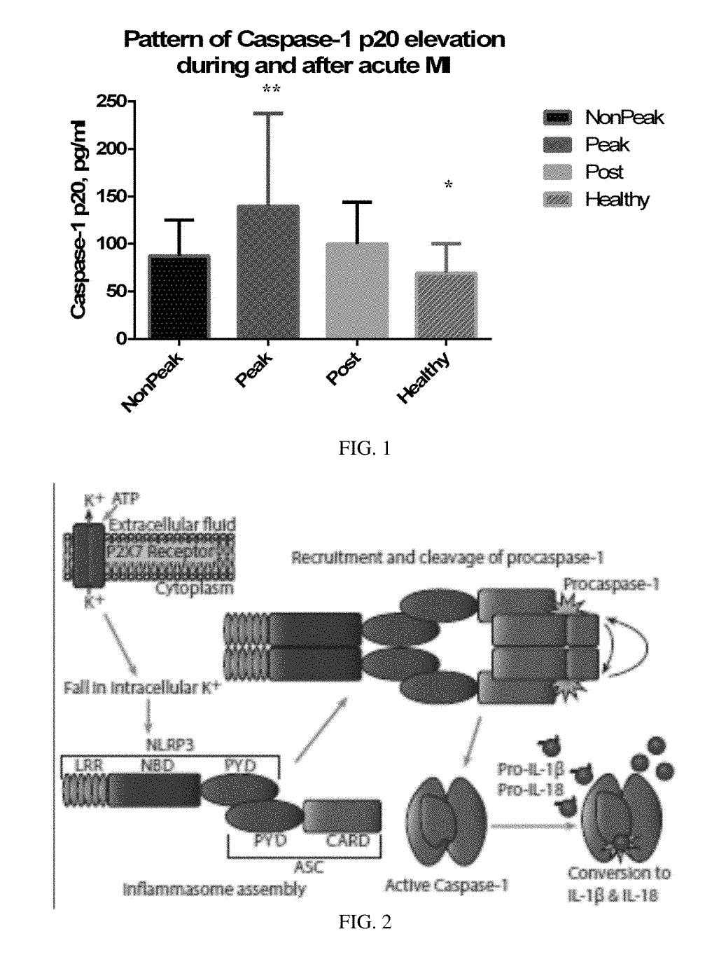

Increased inflammation is associated with vascular disease and contributes to the pathogenesis of AMI. Ongoing inflammation likely also plays a role in the recurrent MI after the initial AMI. Cells dying from ischemic injury release intracellular proteins called danger-associated molecular patterns (DAMPs) to initiate the innate inflammatory response. These molecules bind to the immune Pattern Recognition Receptors (PPRs) on the cell surface to activate the inflammasome in the cytoplasm of cells such as monocytes, macrophages and fibroblasts. Similarly, in vascular disease, inflammasome activation by DAMPs results in a more severe ischemia/reperfusion injury as well as adversely impacting tissue repair. The NLRP3 inflammasome is a cytoplasmic protein complex containing pro-caspase-1 and the ASC protease (apoptosis speck-like protein caspase-recruitment domain), which cleaves pro-caspase-1 into its active p20 and p18 dimers. The NLRP3 inflammasome is activated by a wide variety of signals including DAMPs/PAMPs such as high mobility group proteins, extracellular ATP, uric acids, nucleic acids, and heat shock protein, whole pathogens, or environmental factors such as asbestos. Activated caspase-1 cleaves pro-IL-1b and pro-IL18 to active IL-1b and IL-18 respectively. IL-1b is secreted from monocytes, macrophages, and cardiac fibroblasts and functions to recruit and activate inflammatory leukocytes and delay transdifferentiation of fibroblasts to myofibroblasts. FIG. 2 shows a proposed pathway of activation for Caspase-1 by NLRP3.

While there has been growing pre-clinical evidence for an important role of the NLRP3 inflammasome in atherosclerosis, ischemia/reperfusion injuries, and post-infarction remodeling in animals, very little is known regarding this inflammasome in patients with vascular disease. Since active caspase-1 p20-p18 is proximal to IL-1 β and IL-18, active caspase-1 may be a new marker of inflammation in patients with vascular disease. A previous study investigated the circulating levels of caspase-1, IL-1β, IL-6 and IL-18 in patients with acute MI, focusing on levels pre-MI and several weeks after the acute event. In the present study, we used an enzyme-linked immunoassay to determine the serum level of a cleaved p20 fragment (p20) of caspase-1, the upstream activator of NLRP3 inflammasome in patients with STEMI and NSTEMI. During acute MI, the change in p20 during acute MI was determined with a time course similar to that of the conventional biomarkers of MI cTnI and CK-MB. To ascertain whether p20 level remains elevated after acute MI, its post-MI level was also obtained and then compared to p20 level in healthy subjects. Characterization of caspase-1 levels including its time course during and after AMI can add to our understanding of its role in these patients. The present data showed that serum p20 levels rise and fall during AMI and remains elevated after AMI.

There are growing experimental data in mice demonstrating the importance of NLRP3 inflammasome during tissue injury. The ability to measure and detect this inflammasome activation clinically has made it infeasible to document a role in patients. Our data, for the first time, documents increases in the NLRP3 inflammasome marker caspase-1 p20 peptide during the acute phase of MI. While there is a rise and a fall of p20 during acute MI, similar to those of cTnI and CK-MB, there was no correlation between peak p20 and peak cTnI or peak CK-MB. The reason for lack of any correlation is not clear but may be related to the notion that caspase-1 p20 and cTnI or CK-MB represent markers of inflammation and injury respectively. Caspase-1 p20 is but one inflammatory marker and may not by itself relate to ischemia/reperfusion injury. Experimental evidence for the notion of inflammatory signaling extending ischemic cardiomyocyte injury is less clear. Thus, the lack of correlation between p20 level and cTnI or CK-MB may not be surprising. Overall, our results suggest that the use of this novel assay sets the stage to investigate the circulating p20 level as a new inflammatory marker in patients with vascular disease.

The present study has several limitations, as do all studies that begin new efforts in an important field. We do not know the cellular source of caspase-1 p20 peptide. It is likely multiple cell types are involved in releasing the peptide fragment. The release kinetics of p20 peptide is not known. Information on its release kinetics and half-life may offer better insights into the duration of increased inflammation. Knowledge of the duration of inflammation may provide potential prognostic value of this peptide in circulation. It is of interest that the post-MI p20 peptide level was also higher than that in healthy subjects, likely due to persistent inflammasome activation even after AMI. A heightened inflammatory state may impact adversely on post-infarction remodeling and development of heart failure, a concept suggested by initial evidence for a beneficial effect of the anti-inflammatory agonist colchicine in acute MI.

### CONCLUSION

Caspase-1 can mediate generation of prothrombotic microparticles. Elevated active caspase-1 levels in AMI patients may implicate cleaved caspase-1 in the pathogenesis of AMI and raise the prospect that its serum levels may serve as a new biomarker of vascular instability. The prognostic significance of elevated serum p20 peptide during and after acute MI will require additional study but should be facilitated by the feasible application of the current assay. A larger study with more patients will be needed to answer this potentially important question.

TABLE 1

| | AMI Patients (n = 78) | | |
|---|---|---|---|
| | STEMI | NSTEMI | Statistics |
| | (n = 41) | (n = 37) | (Chi Square) |
| Age | 58.54 ± 1.9* | 66.84 ± 2.4* | |
| Gender | 73% Male, | 68% Male, | 0.588 |
| | 27% Female | 32% Female | |
| Male | 30 | 25 | |
| Female | 11 | 12 | |
| Race | | | |
| African American | 0 | 1 | 0.289 |
| Asian | 0 | 0 | 0.289 |
| Caucasian | 38 | 34 | 0.896 |
| Hispanic | 3 | 2 | 0.731 |
| Avg. Peak Biomarkers | | | |
| CK (μg/mL) | 1546.15 ± 217.36 | 351.38 ± 51.46 | |
| CK-MB (μg/mL) | 237.98 ± 53.43 | 39.59 ± 7.69 | |
| cTnl (μg/mL) | 57.20 ± 10.72 | 8.00 ± 1.94 | |
| Medical History | | | |
| CAD | 9 | 18 | 0.013 |
| HTN | 18 | 27 | 0.009 |
| Dyslipidemia | 19 | 23 | 0.107 |
| Obese | 14 | 11 | 0.676 |
| Medications | | | |
| Beta Blocker | 11 | 18 | 0.046 |
| Aspirin | 12 | 27 | <0.001 |
| ACE Inhibitor | 6 | 23 | <0.001 |
| ARB | 4 | 0 | 0.051 |
| Statin | 13 | 21 | 0.026 |
| Nitrate | 2 | 18 | <0.001 |
| Loop | 1 | 24 | <0.001 |
| Thiazide | 3 | 15 | <0.001 |
| Plavix | 1 | 6 | 0.007 |
| CCB | 4 | 21 | <0.001 |
| Digoxin | 0 | 1 | 0.289 |
| Epogen | 0 | 4 | 0.031 |
| Aldosterone | 0 | 6 | 0.034 |
| Aldoantagonist | | | |
| Smoking History | | | <0.001 |
| Never | 13 | 14 | |
| Former | 11 | 13 | |
| Current | 13 | 7 | |

The demographics and clinical characteristics were obtained from 78 subjects of both the STEMI and NSTEMI patients and were presented in this table (Table 1).
Abbreviations: STEMI = ST Elevation Myocardial Infarction, NSTEMI = Non-ST Elevation Myocardial Infarction, AMI = Acute Myocardial Infarction, CK = Creatine Kinase, CK-MB = Muscle/Brain Creatine Kinase, cTnl = CardiacTroponin, CAD = Coronary Artery Disease, HTN = Hypertension, ARB = Angiotension Receptor Blocker, CCB = Calcium Chanel Blocker.
*STEMI vs NSTEMI Age p = 0.0157.
All Measures are Average ± SEM

### Example 2: Peak Caspase-1 p20 Levels During ST Elevation Myocardial Infarction Correlate with Pre-Existing Hypertension, Age, and Post-Infarction Cardiac Dysfunction

Methods: Between Dec. 10, 2007 and Oct. 23, 2017, 198 eligible subjects diagnosed with acute MI were enrolled in the study. Serial blood samples were collected, two during acute MI and a third during the subject's follow up evaluation, up to two months later. Using a solid phase enzyme-linked immunosorbent assay, the p20 fragment was characterized in patient serum. Cardiac function post-MI was evaluated via standard-of-care echocardiography.

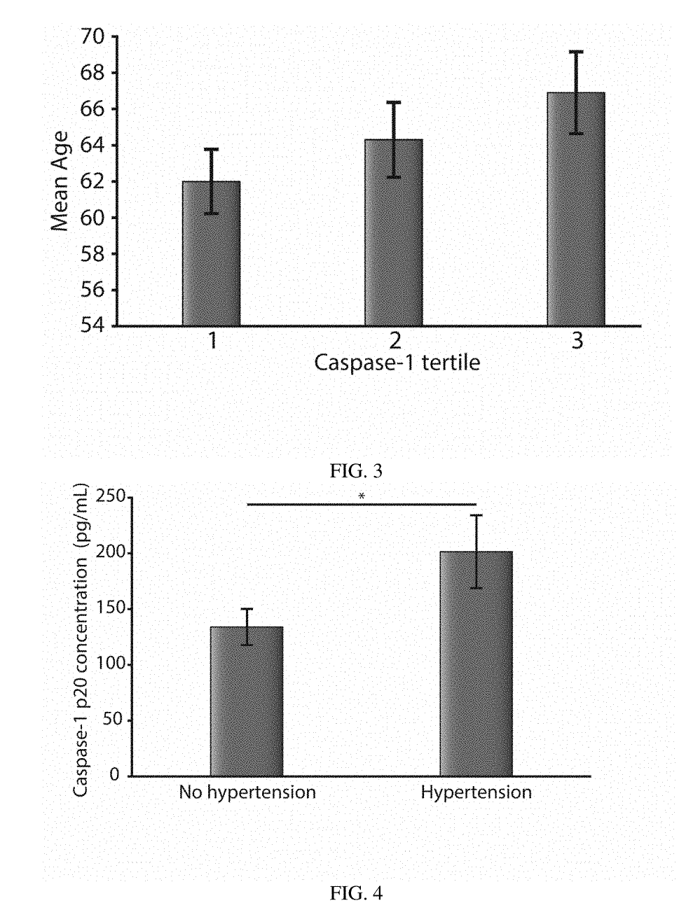

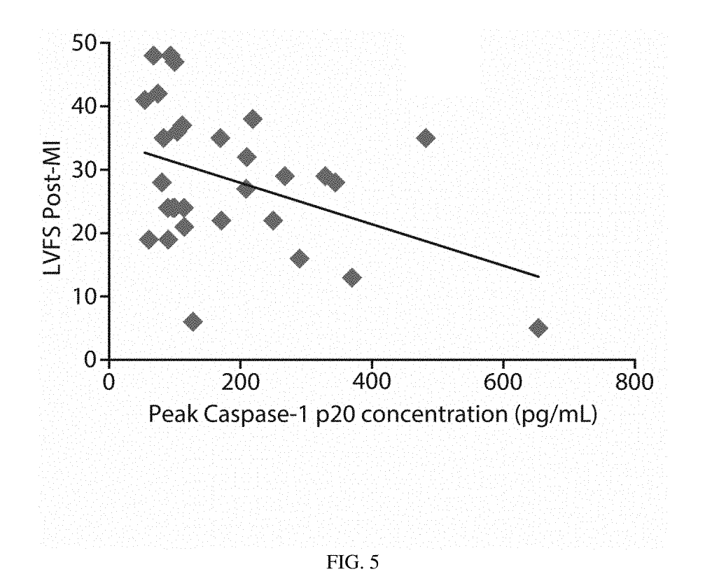

Results: There is a positive correlation between age and peak p20 level during acute MI (P=0.01, linear regression, n=170) as shown in FIG. 3. In a subgroup of STEMI subjects, pre-existing hypertension (HTN) was associated with a 50.3% higher average circulating p20 fragment during acute MI than those without HTN (P=0.0056, Wilcoxon, n=31 per group) as shown in FIG. 4. This was not the case in NSTEMI subjects. In STEMI subjects, the existence of diabetes mellitus or smoking was not associated with higher peak p20 fragment level during acute MI. In a subgroup with acute MI in whom we have post-MI echocardiography, the post-MI cardiac function as quantified by LV fractional shortening via echocardiography was negatively correlated with peak caspase-1 level (P=0.05, Spearman correlation, r=−0.35, n=31) as shown in FIG. 5.

Older subjects have elevated p20 levels during acute MI, consistent with the concept of a larger inflammasome response to MI as age increases. Inflammasome activation is more pronounced in HTN subjects during STEMI, implying a greater degree of inflammatory response in the presence of HTN. Given the increased work required for the heart required of the heart in HTN, it is possible that a greater inflammasome activation contributed to cell death in STEMI. Alternatively, the infarct in STEMI leads to a more exacerbated inflammasome activation in those with pre-existing HTN. The reduced LV fractional shortening in those with higher circulating p20 is consistent with mouse studies showing that Caspase-1 drives greater apoptosis and remodeling following MI with a worse LV function.

The terms “a” and “an” herein do not denote a limitation of quantity, but rather denote the presence of at least one of the referenced item.

All ranges disclosed herein are inclusive and combinable. While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the appended claims.

1. A method for detecting vascular inflammation in an individual, comprising
providing a serum or plasma sample from the individual,
directly or indirectly measuring a level of a caspase-1 proteolytic fragment in the plasma or serum sample from the individual,
comparing the level of the caspase-1 proteolytic fragment in the plasma or serum sample from the individual to a control level in a control sample, and
detecting vascular inflammation in the individual, when the level of the caspase-1 proteolytic fragment in the plasma or serum sample from the individual is elevated by 20% or more compared to the control level in the control sample,
wherein the individual is in need of detection of vascular inflammation.

2. The method of claim 1, wherein the caspase-1 proteolytic fragment is the p-20 fragment.

3. The method of claim 1, wherein the control sample is from a healthy population, from a control individual when the control individual is not experiencing vascular inflammation, or from an individual experiencing an acute myocardial infarction.

4. The method of claim 1, wherein the individual is experiencing or has previously experienced an acute myocardial infarction.

5. The method of claim 4, wherein the acute myocardial infarction is an ST segment elevation myocardial infarction (STEMI).

6. The method of claim 4, wherein the acute myocardial infarction is a non-ST segment elevation myocardial infarction (NSTEMI).

7. The method of claim 1, wherein the individual is experiencing inflammation in an organ that can be damaged via systemic vascular inflammation, wherein the organ is not the heart.

8. The method of claim 1, wherein measuring is by immunoassay with an antibody specific for the caspase-1 fragment.

9. The method of claim 1, wherein measuring is by mass spectrometry.

10. A prognostic method for increased mortality and/or morbidity resulting from acute myocardial infarction in an individual, and/or predicting the recurrence of myocardial infarction in the individual, comprising
providing a serum or plasma sample from the individual,
directly or indirectly measuring a level of a caspase-1 proteolytic fragment in the plasma or serum sample from the individual,
comparing the level of the caspase-1 proteolytic fragment in the plasma or serum sample from the individual to a reference population or a control level in a control sample from healthy subjects, and
detecting an increased risk of mortality and/or morbidity resulting from acute myocardial infarction in the individual wherein the level of the caspase-1 proteolytic fragment in the serum or plasma sample is in the top tertile or quartile of the reference patient population, or wherein the level of the caspase-1 proteolytic fragment in the serum or plasma sample is increased by 20% or more compared to the control level from healthy subjects,
wherein the individual has had an acute myocardial infarction.

11. The method of claim 10, wherein the caspase-1 proteolytic fragment is the p-20 fragment.

12. The method of claim 10, wherein the acute myocardial infarction is an ST segment elevation myocardial infarction (STEMI).

13. The method of claim 10, wherein the acute myocardial infarction is a non-ST segment elevation myocardial infarction (NSTEMI).

14. The method of claim 10, wherein measuring is by immunoassay with an antibody specific for the caspase-1 fragment.

15. The method of claim 10, wherein measuring is by mass spectrometry.

16. A method for determining the success of a therapy for acute myocardial infarction in an individual, comprising
providing a serum or plasma sample from the individual,
directly or indirectly measuring a level of a caspase-1 proteolytic fragment in the plasma or serum sample from the individual,
comparing the level of the caspase-1 proteolytic fragment in the plasma or serum sample from the individual to a reference population or a control level in a control sample from healthy subject, and
detecting an increased risk of mortality and/or morbidity resulting from acute myocardial infarction in the individual wherein the level of the caspase-1 proteolytic fragment in the serum or plasma sample is in the top tertile or quartile of the reference patient population, or wherein the level of the caspase-1 proteolytic fragment in the serum or plasma sample is increased by 20% or more compared to the control level from healthy subjects,
wherein the individual has been treated with therapy for an acute myocardial infarction.

17. The method of claim 16, wherein the caspase-1 proteolytic fragment is the p-20 fragment.

18. The method of claim 16, wherein the acute myocardial infarction is an ST segment elevation myocardial infarction (STEMI).

19. The method of claim 16, wherein the acute myocardial infarction is a non-ST segment elevation myocardial infarction (NSTEMI).

20. The method of claim 16, wherein measuring is by immunoassay with an antibody specific for the caspase-1 fragment.

21. The method of claim 16, wherein measuring is by mass spectrometry.
